(19) United States
(12) Patent Application Publication
(10) Pub. No.: US 2024/0277753 A1
(43) Pub. Date: Aug. 22, 2024

(54) mRNA-based In Vivo Encoding of Darbepoetin Alpha

(71) Applicants: Sarfaraz K. Niazi, Deerfield, IL (US); Matthias Magoola, Kampala (UG); Zafeer Ahmad, Wadsworth, IL (US)

(72) Inventors: Sarfaraz K. Niazi, Deerfield, IL (US); Matthias Magoola, Kampala (UG); Zafeer Ahmad, Wadsworth, IL (US)

(73) Assignee: RNA Therapeutics, Inc., Deerfield, IL (US)

(21) Appl. No.: 18/414,141

(22) Filed: Jan. 16, 2024

(51) Int. Cl. A61K 31/711; A61K 9/127; A61K 9/51; C07K 14/505

(52) U.S. Cl. CPC A61K 31/711; A61K 9/1272; A61K 9/5123; C07K 14/505

(57) ABSTRACT

Darbepoetin alpha treats anemia and it is traditionally manufactured using recombinant Chinese Hamster Ovary cells, a complex and expensive process; this invention reports an mRNA composition that encodes darbepoetin alpha in vivo as a therapeutic protein.

### SEQUENCE LISTING

The instant application contains a Sequence Listing, which has been submitted electronically in ASCII format and is hereby incorporated by reference in its entirety. Said ASCII text file, created on Jan. 17, 2024, is named 27134_18414141_01-17-2024 CRFE, and is 300 kb in size.

### BACKGROUND OF THE INVENTION

Darbepoetin alfa (INN) is a re-engineered form of erythropoietin containing 5 amino acid changes (N30, T32, V87, N88, T90) resulting in the creation of 2 new sites for N-linked carbohydrate addition. It has a 3-fold longer serum half-life than epoetin alpha and epoetin beta. It stimulates erythropoiesis (increases red blood cell levels) by the exact mechanism as rHuEpo (binding and activating the EPO receptor). It is used to treat anemia, commonly associated with chronic kidney failure and cancer chemotherapy. Amgen markets Darbepoetin under the trade name Aranesp, which was approved in September 2001 by the US Food and Drug Administration for treating anemia in patients with chronic kidney failure by intravenous or subcutaneous injection. In June 2001, it was approved by the European Medicines Agency for this indication and the treatment of anemia in cancer patients undergoing chemotherapy.

Darbepoetin is produced by recombinant DNA technology in modified Chinese hamster ovary cells. It differs from endogenous erythropoietin (EPO) by containing two more N-linked oligosaccharide chains. It is an erythropoiesis-stimulating 165-amino acid protein. There are no biosimilar products approved by the FDA for Aranesp. It is on the World Health Organization's List of Essential Medicines.

The present invention is an mRNA product that expresses darbepoetin in vivo, leading to a comparable concentration profile as observed in the administration of Aranesp.

### SUMMARY OF THE INVENTION

The heart of the mRNA is the Coding Sequence, comprising codons, which are nucleotide triplets that dictate the amino acid sequence in the resulting protein. Following the coding sequence is the 3′ Untranslated Region (3′ UTR), which, like its 5′ counterpart, doesn't code for protein but plays a role in mRNA stability and translation regulation. Finally, the Poly-A Tail, a string of adenine nucleotides at the mRNA's 3′ end, further stabilizes the mRNA and influences its lifespan for translation, assisting in its transport from the nucleus to the cytoplasm. Additionally, Ribosome Binding Sites, primarily located within the 5′ UTR, are critical for correct ribosome assembly and translation initiation on the mRNA.

The number of protein molecules generated from a single mRNA is primarily determined by “translation efficiency.” The stability of the mRNA molecule, the availability of different translation components, and the existence of translation initiation sites are some factors affecting translation efficiency.

The length of the mRNA, translation efficiency, and stability of the resultant protein all affect how many protein molecules can be translated from a single mRNA molecule. It is noteworthy that translation is a dynamic process and that a chain of ribosomes known as polysomes can be formed when multiple ribosomes simultaneously translate the same mRNA molecule. Various ribosomes can translate a single mRNA molecule; this phenomenon is known as polysome or ribosome “clustering.” This makes it possible to produce several protein molecules from the same mRNA template effectively and concurrently. Several factors, including ribosome availability, cellular circumstances, and particular mRNAs and their associated regulatory elements, govern how many ribosomes can translate mRNA simultaneously. The process of developing mRNA encoding mRNAs comprises several well-defined but complex steps:
Select the sequence: Start by identifying a specific sequence of the expressed.
Design mRNA Sequence: Design an mRNA sequence that encodes the selected protein. The mRNA sequence should follow the rules of mRNA transcription, such as starting with a 5′ cap and including a 3′ poly-A tail. Ensure the sequence is in-frame with the ribosome so that translation produces the desired epitope.
Codon Optimization: Optimize the mRNA sequence for translation efficiency in the desired host cell. This may involve choosing more frequently used codons in the host organism to ensure efficient translation.
Consider mRNA Modifications: To enhance stability and translation efficiency, consider incorporating modified nucleotides, such as pseudouridine or 5-methylcytidine, into the mRNA sequence. These modifications can improve mRNA stability and reduce immune recognition. Also significant is the replacement of uridine with pseudouridine.
Delivery Method: Determine how to deliver the mRNA to the target cells. This can include electroporation, lipid nanoparticles, or viral vectors.
Expression System: Choose an appropriate expression system for producing pDNA.
In Vitro Translation: Transcribe and translate the mRNA in an in vitro translation system, such as a cell-free translation system or using cultured cells. This will help verify that the mRNA is producing the desired epitope. mRNA products utilize in vivo ribosomes to express proteins.

The next step is to convert the protein sequences into nucleoside coding sequences by first converting the target polypeptide sequence into DNA through reverse transcription and then to RNA. First, multiple epitopes from the same protein or its isoforms can be linked together using linkers including but not limited to various combinations of Alanine, Asparagine, Glutamic Acid, Glycine, Leucine, Lysine, Phenylalanine, Proline, Serine, and Threonine, or combinations thereof, using singularly or as repeated groups.

mRNA product functional regulation requires untranslated regions (UTRs) between the open reading frame (ORF) and the 5′ and 3′ ends, upstream and downstream of the mRNA. These UTRs contain regulatory sequences associated with mRNA stability and efficient and correct mRNA translation. They also help recognize mRNA by ribosomes and help in post-transcriptional modification of the mRNA. The mRNA translation and its half-life can be improved by including cis-regulatory sequences in the UTRs. Additionally, the inclusion of naturally occurring sequences, such as those derived from alpha- and beta-globins.

mRNA molecules are large (104-106 Da) and negatively charged. They are unable to pass through the lipid bilayer of cell membranes. Naked mRNA would be destroyed and degraded by the nucleases in the bloodstream. In addition, naked mRNA is also attached and engulfed by immune cells in the tissue and the serum. Methods to deliver mRNA molecules into the cells include gene guns, electroporation, and ex vivo transfection. The in vivo methods of delivering mRNA involve the transfection of immune or non-immune cells using lipids or transfecting agents.

Although naked mRNA, liposomes, and polyplexes have shown clinical effectiveness in humans, LNPs for mRNA encoding mRNAs are the only drug delivery system that has demonstrated clinical efficacy and has been approved for human use. The COVID-19 mRNA vaccines against SARS-COV-2, developed by Moderna and Pfizer/BioNTech, employ LNPs to deliver the mRNA payload to the body. LNPs are currently the foremost non-viral delivery vector employed for gene therapy. The clinical effectiveness of LNPs was first demonstrated when LNP-siRNA therapeutic Onpattro® (patisiran) was approved by the US FDA for hereditary transthyretin-mediated amyloidosis. LNP formulations are the most successful, effective, and safe method of delivery of mRNA encoding mRNAs for human immunizations. LNPs offer numerous advantages for mRNA delivery to the site of action, including ease of formulation and scale-up, highly efficient transfection capacity, low toxicity profile, modularity, compactivity with different nucleic acid types and sizes, protection of mRNA from internal degradation, and increasing the half-life of mRNA encoding mRNAs. LNPs are typically composed of four components: an ionizable cationic lipid, a helper phospholipid, cholesterol, and a PEGylated lipid. These lipids encapsulate the mRNA encoding mRNA's payload and protect the nucleic acid core from degradation.

### DETAILS OF THE INVENTION

Messenger RNA (mRNA) is crucial in translating genetic information from DNA into proteins. It includes several key components, each with a specific function. The 5′ Cap, a modified guanine nucleotide at the mRNA's 5′ end, ensures mRNA stability and aids in translation initiation, protecting the mRNA from degradation and assisting in ribosome recognition. Adjacent to the cap is the 5′ Untranslated Region (5′ UTR), a sequence not coding for protein but crucial in regulating translation efficiency and ribosome binding.

Advantageously, mRNA can be manufactured in a large-scale fashion and enables the production of a robust immune response based on mRNA encoding, for example, antigens that produce antibodies specific to proteins of the target infecting cell.

In various embodiments, the coding mRNA comprises, preferably in 5′- to 3′-direction, the following elements:
a) 5′-cap structure selected from m7G(5′), m7G(5′)ppp(5′)(20MeA), or m7G(5′)ppp(5′)(2′OMeG);
b) A 5′-terminal start element is selected from AUG, GUG, and UUG.
c) Optionally, a signal peptide
d) An open reading frame to encode darbepoetin alpha
e) A stop codon UAA, UAG, or UGA
f) 3-UTR
g) poly(A) sequence comprising about 50 to about 250 adenosines.

The 5′ end of the mRNA contains a 7-methylguanosine (m7G) moiety, followed by a triphosphate moiety to the first nucleotide (m7GpppN). m7GpppN is called a 5′ cap, a protective structure that protects RNA from exonuclease cleavage, regulates pre-mRNA splicing, and initiates mRNA translation and nuclear export of the mRNA to the cytoplasm.

The mRNA can be modified to improve its efficacy and stability by introducing many post-transcriptional modifications. Some of these include 2′-O-methylation at position 2′ of the ribose ring at the first nucleotide (Cap 1, m7GpppN1m) and the second nucleotide (Cap 2, m7GpppN1mN2m) as well. These modifications in the 5′ cap structure not only increase the translation efficiency of mRNA but also stop the activation of endosomal and cytosolic receptors, including RIG-I and MDA5, which act as defensive mechanisms against viral mRNA.

Hence, the 2′-O-methylation of the 5′ cap structure is a highly desirable property for increasing and enhancing the protein production from the mRNA after its transcription and blocking any undesirable immune responses from the host immune system to the antigenic IVT mRNA. This 5′ cap can be achieved by adding S-adenosyl methionine and the Cap 0 structure to the IVT mRNA reaction, which yields IVT mRNA with the Cap 1 structure and S-adenosyl-L-homocysteine. Cap 1 refers to m7GpppNm, where Nm represents any nucleotide with a 2′O methylation. This structure plays a crucial role in RNA stability and the initiation of protein synthesis. m7G represents a 7-methylguanosine residue. It's a modified guanine nucleotide with a methyl group attached to the nitrogen at the 7th position. This modification is crucial for RNA stability and efficient translation; ppp is a triphosphate bridge. It connects the 5′ end of the mRNA with the m7G cap. This linkage is unusual because it's a 5′-to-5′ triphosphate linkage, unlike the typical 5′-to-3′ phosphodiester bonds in RNA; Am signifies a 2′-O-methyladenosine residue. It's a modification where a methyl group is added to the 2′ hydroxyl group of the first nucleotide of the mRNA adjacent to the cap. This modification can enhance the stability of the mRNA and also plays a role in distinguishing self-RNA (e.g., from a cell's genes) from non-self-RNA (e.g., viruses or other pathogens) in the immune response.

The cap1 structure (m7GpppAm) is a common feature in eukaryotic mRNA and is essential for various aspects of RNA metabolism, including RNA stability, export from the nucleus, and translation initiation. It also helps recognize the mRNA by the ribosome and other components of the translation machinery. In the modified structure, there's an additional methyl group at the 3′ position of the m7G cap (m7G+m3′). This modification might further influence the interaction of the cap with cellular proteins and potentially affect mRNA stability and translation efficiency.

An example of the modified 5′-cap1 structure (m7G+m3′-5′-ppp-5′-Am) can be found in certain messenger RNAs (mRNAs) used in mRNA-based vaccines, such as those developed for COVID-19. In these vaccines, the mRNA carries the instructions to produce a specific viral protein (like the spike protein of the SARS-COV-2 virus) that triggers an immune response in the body. The modified cap structure plays a crucial role in these mRNA molecules.

The various types of CAPs that can be beneficial in the present invention include ARCA, Bridged Cap (BCAP), Cap0, Cap1, Cap2, Cap3, Cap4, CleanCap, Hypermodified Caps, Modified Cap1, Synthetic or Designer Caps, Tobacco Mosaic Virus (TMV) Cap, and

In preferred embodiments, the cap1 structure of the coding mRNA of the invention is formed using co-transcriptional capping using tri-nucleotide cap analogs m7G(5′)ppp(5′)(2′OMeA)pG or m7G(5′)ppp(5′)(2′OMeG)pG. A preferred cap1 analog that may suitably be used in manufacturing the coding mRNA of the invention is m7G(5′)ppp(5′)(2′OMeA)pG.

In mRNA therapeutics, heterologous peptides are often used as signal sequences to direct the synthesized protein to specific locations within the cell, such as the endoplasmic reticulum (ER) or extracellular space. These signal peptides play a crucial role in ensuring that the protein encoded by the mRNA reaches its intended destination to function effectively. Some commonly used heterologous signal peptides in mRNA therapeutics include:
a) Human Ig kappa chain signal peptide (IgK): Derived from the immunoglobulin kappa light chain, this signal peptide is widely used to direct the expressed protein to the secretory pathway.
b) Honeybee melittin signal peptide: Originally from the honeybee venom, the melittin signal sequence is known for its efficiency in directing proteins for secretion.
c) Tissue plasminogen activator signal peptide (tPA): The tPA signal sequence is commonly used to process proteins efficiently. It is derived from the human tissue plasminogen activator.
d) Granulocyte-macrophage colony-stimulating factor signal peptide (GM-CSF): Taken from the GM-CSF, this signal peptide is used to direct proteins to the extracellular space or the secretory pathway.
e) Albumin signal peptide: This signal sequence, derived from human serum albumin, guides proteins towards secretion.
f) Signal peptide of the human erythropoietin (EPO): The EPO signal peptide is used in certain therapeutic applications where efficient secretion of the protein is desired.
g) Secretory Pathway Signal Peptide (Sec/SPI): A common signal peptide directing proteins to the secretory pathway.
h) Preproinsulin Signal Peptide: Derived from preproinsulin, this signal peptide is used for insulin analogs and other therapeutic proteins requiring secretion.
i) Alpha Factor Signal Sequence (from Saccharomyces cerevisiae): Commonly used in yeast expression systems to direct protein secretion.
j) Basic Secretory Signal Peptide (BSSP): A synthetic signal peptide designed for efficient protein secretion.
k) Endothelin Signal Peptide: Used for directing proteins to the secretory pathway, particularly in cardiovascular therapeutics.
l) Interferon-alpha Signal Peptide: Employed in producing type I interferon and related proteins.
m) Lysozyme Signal Peptide: Derived from lysozyme, this peptide directs proteins to the secretory pathway in various expression systems.
n) Calreticulin Signal Sequence: Used for targeting proteins to the endoplasmic reticulum.
o) Vascular Endothelial Growth Factor (VEGF) Signal Peptide: Used in angiogenesis and tissue regeneration therapies.
p) Beta-Lactamase Signal Peptide: Often used in bacterial expression systems for protein secretion.
q) p67 Signal Sequence (from Trypanosoma brucei): Employed in certain therapeutic applications for targeting proteins to specific cellular compartments.
r) Glucagon Signal Peptide: Used in the expression of glucagon-like peptides and analogs.

The invention's open reading frame or coding sequence of mRNA can be prepared using any method known in the art, including chemical synthesis, such as, e.g. solid phase mRNA synthesis, and in vitro methods, such as mRNA in vitro transcription reactions.

In a preferred embodiment, the coding mRNA is obtained by RNA in vitro transcription.

Examples of 5′-UTR sourcing comprise Arabidopsis thaliana Light-Harvesting Chlorophyll a/b-binding Protein (LHCB) 5′ UTR, C. elegans unc-54 5′ UTR, Drosophila melanogaster Hunchback (hb) mRNA 5′ UTR, E. coli lac operon 5′ UTR, Frog (Xenopus laevis) Oocyte maturation factor mRNA 5′ UTR, Human Insulin mRNA 5′ UTR, Human TP53 (Tumor Protein p53) mRNA 5′ UTR, Human β-globin mRNA 5′ UTR, Maize (Zea mays) Photosynthetic enzyme mRNA 5′ UTR, Mouse α-actin mRNA 5′ UTR, Rice (Oryza sativa) Chlorophyll a/b-binding protein (CAB) mRNA 5′ UTR, Tomato (Solanum lycopersicum) Fruit-ripening gene 5′ UTR, Yeast (Saccharomyces cerevisiae) Heat Shock Protein (HSP) mRNA 5′ UTR, Yeast Alcohol Dehydrogenase (ADH1) 5′ UTR, and Zebrafish Myogenic Factor 5 (myf5) 5′ UTR.

In embodiments, the selection of the components of an mRNA is based on experimental confirmation that the claimed sequence encodes the target protein of Sequence No. 01 Seq. No. 2 is the preferred signal peptide. Sequence No. 3 is the preferred CRF with U replaced with T for the purpose of creation ST.26 listing.

| # | Sequence |
|---|---|
| 1. | APPRLICDSRVLERYLLEAKEAENITTGCNETCSLNENITVPDTK |
| | VNFYAWKRMEVGQQAVEVWQGLALLSEAVLRGQALLVNSSQVNET |
| | LQLHVDKAVSGLRSLTTLLRALGAQKEAISPPDAASAAPLRTITA |
| | DTFRKLFRVYSNFLRGKLKLYTGEACRTGDR |
| 2. | MGWSCIILFLVATATGVHS |
| 3. | ATGGGCTGGAGCTGCATTATTCTGTTTCTGGTGGCGACCGCGAC |
| | CGGCGTGCATAGCGCGCCGCCGCGCCTGATTTGCGATAGCCGC |
| | GTGCTGGAACGCTATCTGCTGGAAGCGAAAGAAGCGGAAAAC |
| | ATTACCACCGGCTGCAACGAAACCTGCAGCCTGAACGAAAACA |
| | TTACCGTGCCGGATACCAAAGTGAACTTTTATGCGTGGAAACG |
| | CATGGAAGTGGGCCAGCAGGCGGTGGAAGTGTGGCAGGGCCT |
| | GGCGCTGCTGAGCGAAGCGGTGCTGCGCGGCCAGGCGCTGCTG |
| | GTGAACAGCAGCCAGGTGAACGAAACCCTGCAGCTGCATGTGG |
| | ATAAAGCGGTGAGCGGCCTGCGCAGCCTGACCACCCTGCTGCG |
| | CGCGCTGGGCGCGCAGAAAGAAGCGATTAGCCCGCCGGATGC |
| | GGCGAGCGCGGCGCCGCTGCGCACCATTACCGCGGATACCTTT |
| | CGCAAACTGTTTCGCGTGTATAGCAACTTTCTGCGCGGCAAACT |
| | GAAACTGTATACCGGCGAAGCGTGCCGCACCGGCGATCGC |

The encoded protein has the signal peptide (Sequence No. 2) that breaks off after assisting in the surface transport of the molecule.

In embodiments, the nucleotide mixture used in mRNA in vitro transcription may additionally contain modified nucleotides as defined herein. Modifying codons, particularly in codon optimization, involves various strategies to enhance gene expression or protein synthesis in a target organism. In codon optimization, structural changes to the RNA sequence are often made to enhance the stability and efficiency of mRNA translation. These changes are designed to avoid hindrances in the translation process and improve overall protein expression.

Altering sequences that form stable hairpin or stem-loop structures in the mRNA. For instance, a sequence like GGGGGG, which might form a strong secondary structure, could be altered to a less self-complementary sequence like GAGAGA without changing the amino acid sequence.

Modifying the GC content of the mRNA to optimize stability and efficiency. High GC content can lead to secondary solid structures, while low GC content might reduce mRNA stability. Adjustments are made to reach an optimal balance. For example, replacing AT-rich codons with GC-rich synonymous codons or vice versa.

Removing or altering sequences that are known to signal for rapid mRNA degradation. For example, specific sequences like AU-rich elements in eukaryotes might be modified to increase the half-life of the mRNA.

Changing sequences can cause the ribosome to stall during translation. For instance, a stretch of rare codons or a sequence that forms a tight secondary structure might be modified to ensure smooth progression of the ribosome.

Altering sequences that could mimic regulatory elements like promoters, enhancers, or internal ribosome entry sites (IRES) could interfere with proper transcription and translation.

Adjusting codon usage to match the tRNA pool of the host organism. Overusing a particular codon can deplete its corresponding tRNA, slowing translation. The sequence is modified to use more abundant tRNAs.

Reducing repetitive sequences that can lead to recombination events or genomic instability. This also helps in avoiding slippage during transcription or translation.

These structural changes are tailored to the specific requirements of the host organism and the protein being expressed. The goal is to create an mRNA sequence that is efficiently translated with minimal interruptions or instability, leading to higher protein yields.

In that context, preferred modified nucleotides comprise pseudouridine, N1-methylpseudouridine, 5-methylcytosine, and 5-methoxyuridine. Embodiments of uracil nucleotides in the nucleotide mixture are replaced (either wholly or partially) by pseudouridine and/or N1-methyl pseudouridine to obtain a modified coding mRNA.

In preferred embodiments, the nucleotide mixture (i.e., the fraction of each nucleotide in the mix) used for mRNA in vitro transcription reactions may be optimized for the given mRNA sequence.

In a further preferred embodiment, the coding mRNA, particularly the purified coding mRNA, is lyophilized. The mRNA of the invention, particularly the purified mRNA, may also be dried using spray-drying or spray-freeze drying.

A second aspect relates to an encoding mRNA comprising at least one coding mRNA of the first aspect.

Notably, embodiments relating to the encoding mRNA of the second aspect may likewise be read on and be understood as suitable embodiments of the encoding mRNA of the third aspect. Also, embodiments relating to the encoding mRNA of the third aspect may likewise be read on and be understood as suitable embodiments of the encoding mRNA of the second aspect (comprising the mRNA of the first aspect). In preferred embodiments of the second aspect, said encoding mRNA comprises at least one mRNA encoding peptides or proteins according to the first aspect, or an immunogenic fragment or immunogenic variant thereof, wherein said encoding mRNA is to be, preferably, administered intramuscularly or intradermal.

Preferably, intramuscular or intradermal administration of the said encoding mRNA results in the expression of the encoded antigen in a subject. Preferably, the encoding mRNA of the second aspect is suitable for an ideal encoding mRNA.

The encoding mRNA may comprise a safe and effective amount of the mRNA to result in the encoded antigenic protein's expression and activity. At the same time, a “safe and effective amount” is small enough to avoid serious side effects.

In the context of the invention, an “encoding mRNA” refers to any type of encoding mRNA in which the specified ingredients (e.g., mRNA encoding proteins or peptides, e.g., in association with a polymeric carrier or LNP) may be incorporated, optionally along with any further constituents, usually with at least one pharmaceutically acceptable carrier or excipient. The encoding mRNA may be a dry encoding mRNA, such as a powder or granules, or a solid unit, such as a lyophilized form. Alternatively, the encoding mRNA may be liquid, and each constituent may be independently incorporated in dissolved or dispersed (e.g., suspended or emulsified) form.

In a preferred embodiment of the second aspect, the encoding mRNA comprises mRNA coding at least one protein or peptide and, optionally, at least one pharmaceutically acceptable carrier or excipient.

In particularly preferred embodiments of the second aspect, the encoding mRNA comprises at least one coding mRNA, wherein the coding mRNA includes or consists of an mRNA sequence that is identical or at least 70% to 99% to a nucleic acid sequence selected from the group consisting of epitopes chosen, and, optionally, at least one pharmaceutically acceptable carrier or excipient.

The term “pharmaceutically acceptable carrier” or “pharmaceutically acceptable excipient” as used herein preferably includes the liquid or non-liquid basis of the encoding mRNA for administration. If the encoding mRNA is liquid, the carrier may be water, e.g., pyrogen-free water; isotonic saline or buffered (aqueous) solutions, e.g., phosphate, citrate, etc. buffered solutions.

Furthermore, organic anions of the cations may be in the buffer. Accordingly, in embodiments, the mRNA encoding mRNA of the invention may comprise pharmaceutically acceptable carriers or excipients using one or more pharmaceutically acceptable carriers or excipients to, e.g., increase stability, increase cell transfection, permit the sustained or delayed, increase the translation of encoded epitopes. In addition to traditional excipients such as all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers, diluents, or encapsulating compounds, which are suitable for administration to a subject, may also be used.

The term “compatible,” as used herein, means that the constituents of the encoding mRNA are capable of being mixed with mRNA and, optionally, a plurality of mRNAs of the encoding mRNA in such a manner that no interaction occurs, which would substantially reduce the biological activity or the pharmaceutical effectiveness of the encoding mRNA under typical use conditions (e.g., intramuscular, or intradermal administration).

At least one pharmaceutically acceptable carrier or excipient of the encoding mRNA may preferably be selected to be suitable for intramuscular or intradermal delivery. Accordingly, the encoding mRNA is preferably a pharmaceutical encoding mRNA suitable for intramuscular or intradermal administration.

The pharmaceutical encoding mRNA is contemplated for use, but is not limited to, humans and other primates; mammals, including commercially relevant mammals such as cattle, pigs, horses, sheep, cats, dogs, mice, and rats; and birds, including commercially relevant birds such as poultry, chickens, ducks, geese, and turkeys.

Pharmaceutical encoding mRNAs of the present invention may suitably be sterile and pyrogen-free. Furthermore, one or more compatible solid or liquid filler diluents or encapsulating compounds, which are suitable for administration to a person, may also be used. The term “compatible,” as used herein, means that the constituents of the encoding mRNA are capable of being mixed with mRNA and, optionally, the further coding mRNA of the encoding mRNA in such a manner that no interaction occurs, which would substantially reduce the biological activity or the pharmaceutical effectiveness of the encoding mRNA under typical use conditions.

In embodiments, the encoding mRNA, as defined herein, may comprise a plurality of or at least more than one of the coding mRNA species as defined in the context of the first aspect of the invention.

In a preferred embodiment of the second aspect, the coding mRNA is complexed or associated with or at least partially complexed or partially associated with one or more cationic or polycationic compounds, preferably cationic or polycationic polymer, cationic or polycationic polysaccharide, cationic or polycationic lipid, cationic or polycationic protein, cationic or polycationic peptide, or any combinations thereof.

Coding mRNA is complexed with protamine in a preferred embodiment of the second aspect. In this context, it is particularly preferred that coding mRNA is complexed or at least partially complexed with a cationic or polycationic compound and a polymeric carrier, preferably cationic proteins or peptides.

In a preferred embodiment of the second aspect, coding mRNA is complexed or partially complexed, with at least one cationic or polycationic protein, peptide, or a combination thereof.

According to embodiments, the encoding mRNA of the present invention comprises the coding mRNA as defined in the context of the first aspect and a polymeric carrier.

A suitable polymeric carrier may be a polymeric carrier formed by disulfide-crosslinked cationic compounds. The disulfide-crosslinked cationic compounds may be the same or different from each other. The polymeric carrier can also contain further components.

The polymeric carrier used in the present invention may comprise mixtures of cationic peptides, proteins, polymers, and optionally further components as defined herein, which are crosslinked by disulfide bonds (via —SH groups).

In a particularly preferred embodiment, the polymeric carrier is a peptide polymer, preferably a polyethylene glycol/peptide polymer as defined above, and a lipid component, preferably a lipidoid component. In a preferred embodiment of the second aspect, coding mRNA of the first aspect is complexed or associated with a polymeric carrier, preferably with a polyethylene glycol/peptide polymer as defined above, and a lipidoid component, wherein the lipidoid component is a compound.

In preferred embodiments of the second aspect, coding mRNA is complexed, encapsulated, partially encapsulated, or associated with one or more lipids (e.g., cationic lipids and/or neutral lipids), thereby forming liposomes, lipid nanoparticles (LNPs), lipoplexes, and nanoliposomes.

The liposomes, lipid nanoparticles (LNPs) lipoplexes, and nanoliposomes with incorporated mRNA may be entirely or partially located in the interior space of the liposomes, lipid nanoparticles (LNPs), lipoplexes, and nanoliposomes within the membrane, or associated with the exterior surface of the membrane. Incorporating nucleic acid into liposomes is also referred to herein as “encapsulation,” wherein the mRNA is entirely contained within the interior space of the liposomes, lipid nanoparticles (LNPs), lipoplexes, and nanoliposomes. The purpose of incorporating an mRNA into liposomes, lipid nanoparticles (LNPs), lipoplexes, and nanoliposomes is to protect the mRNA from an environment that may contain enzymes or chemicals that degrade mRNA and systems or receptors that cause the rapid excretion of the mRNA. Moreover, incorporating an mRNA into liposomes, lipid nanoparticles (LNPs), lipoplexes, and nanoliposomes may promote the uptake of the mRNA and, hence, enhance the therapeutic effect of the mRNA-encoding antigenic peptides. Accordingly, incorporating an mRNA into liposomes, lipid nanoparticles (LNPs), lipoplexes, and nanoliposomes may be particularly suitable for encoding mRNA, e.g., intramuscular or intradermal administration.

In this context, “complexed” or “associated” refers to the essentially stable combination of coding mRNA of the first aspect with one or more lipids into larger complexes or assemblies without covalent binding.

According to further embodiments, the encoding mRNA of the second aspect may comprise at least one adjuvant. Suitably, the adjuvant is preferably added to enhance the immunostimulatory properties of the encoding mRNA.

The encoding mRNA of the second aspect may comprise, besides the components specified herein, at least one further component, which may be selected from the group consisting of further antigens (e.g., in the form of a peptide or protein) or further antigen-encoding nucleic acids, a further immunotherapeutic agent; one or more auxiliary substances (cytokines, such as monokines, lymphokines, interleukins or chemokines); or any further compound, which is known to be immune stimulating due to its binding affinity (as ligands) to human Toll-like receptors; and an adjuvant nucleic acid, preferably an immunostimulatory RNA (isRNA), e.g., CpG-RNA, etc.

The LNP formulation is also an adjuvant.

Thirdly, the invention provides an encoding of darbepoetin alpha. The encoding mRNA comprises the coding RNA of the first aspect and, optionally, the encoding mRNA of the second.

Notably, embodiments relating to the encoding mRNA of the second aspect may likewise be read on and be understood as suitable embodiments of the encoding mRNA of the third aspect. Also, embodiments relating to the encoding mRNA of the third aspect may likewise be read on and be understood as suitable embodiments of the encoding mRNA of the second aspect (comprising the RNA of the first aspect).

As a pharmaceutical, the encoding mRNA can be used according to the invention for human medical and veterinary medical purposes (mammals, vertebrates, avian species).

Further, the present invention relates to the first medical use of the coding RNA of the first aspect, the second aspect encoding mRNA, and the third encoding mRNA.

Accordingly, the RNA of the first aspect, the encoding mRNA of the second aspect, and the encoding mRNA of the third aspect are used as a medicament.

The present invention provides several applications and uses of the coding RNA of the first aspect, the encoding mRNA of the second, or the encoding mRNA of the third.

RNA encoding mRNA, as an encoding mRNA, may be used for human medical and veterinary medical purposes, preferably for human medical purposes.

In embodiments, the RNA of the first aspect, the encoding mRNA of the second aspect, and the encoding mRNA of the third aspect are used to treat anemia.

The encoding mRNA or the encoding mRNA defined herein may preferably be administered locally. An intradermal, subcutaneous, intranasal, or intramuscular route may administer encoding mRNAs or encoding mRNAs. Inventive encoding mRNAs or encoding mRNAs of the invention are, therefore, preferably formulated in liquid (or sometimes solid) form. Conventional needle or needle-free jet injection may administer the inventive encoding mRNA in embodiments. Preferred in that context is the RNA, the encoding mRNA, and the encoding mRNA administered by intramuscular needle injection.

An mRNA sequence for an antibody against TNF-alpha is produced by selected epitopes and/or peptides, wherein the epitopes and peptides can be optionally linked as a single chain.

1. An mRNA encoding darbepoetin alpha protein in vivo to treat anemia comprising
a) at least one 5′-cap;
b) at least one heterologous 5′ untranslated region (5′-UTR);
c) at least one heterologous 3′ untranslated region (3′-UTR);
d) at least one heterologous signal peptide;
e) at least one poly(A) tail comprising 30 to 150;
e) at least one coding reading frame (CRF) is needed to encode darbepoetin alpha.

2. The mRNA encoding darbepoetin alpha protein in vivo of claim 1, wherein the darbepoetin alpha sequence comprises Sequence No 1.

3. The mRNA encoding darbepoetin alpha protein in vivo of claim 1, wherein the signal peptide comprises Sequence No 2.

4. The mRNA encoding darbepoetin alpha protein in vivo of claim 1, wherein the Coding Reading Frame comprises Sequence No 3.

5. The mRNA encoding darbepoetin alpha protein in vivo of claim 1, wherein RNA sequences are codon-optimized by replacing uridine either wholly or partially by pseudouridine and N1-methyl pseudouridine or a combination thereof.

6. The mRNA encoding darbepoetin alpha protein in vivo of claim 1, wherein the coding mRNA is a self-replicating RNA, a circular RNA, or a replicon RNA.

7. The mRNA encoding darbepoetin alpha protein in vivo claim 1, wherein the coding mRNA optionally comprises at least one pharmaceutically acceptable carrier.

8. The mRNA encoding darbepoetin alpha protein in vivo of claim 1, wherein the coding mRNA is complexed or associated with or at least partially complexed or partially associated with one or more cationic or polycationic compounds, preferably cationic or polycationic polymer, cationic or polycationic polysaccharide, cationic or polycationic lipid, cationic or polycationic protein, cationic or polycationic peptide, or any combinations thereof.

9. The mRNA encoding darbepoetin alpha protein in vivo of claim 1, wherein the coding mRNA is complexed or associated with one or more lipids, thereby forming liposomes, lipid nanoparticles (LNP), lipoplexes, and nanoliposomes, presented with or without lyophilization.

10. The mRNA encoding darbepoetin alpha protein in vivo of claim 9, wherein the LNP essentially consists of
(i) at least one cationic lipid;
(ii) at least one neutral lipid;
(iii) at least one steroid or steroid analog; and
(iv) at least once a PEG-lipid.


SEQUENCE LISTING (3 sequences)

SEQ ID NO: 1 - protein, 166 aa, Homo sapiens
APPRLICDSRVLERYLLEAKEAENITTGCNETCSLNENITVPDTKVNFYAWKRMEVGQQAVEVWQGLALLSEAVLRGQALLVNSSQVNETLQLHVDKAVSGLRSLTTLLRALGAQKEAISPPDAASAAPLRTITADTFRKLFRVYSNFLRGKLKLYTGEACRTGDR

SEQ ID NO: 2 - protein, 19 aa, Homo sapiens
MGWSCIILFLVATATGVHS

SEQ ID NO: 3 - RNA, 555 nt, Homo sapiens
  source 1..555: mol_type = mRNA
atgggctggagctgcattattctgtttctggtggcgaccgcgaccggcgtgcatagcgcgccgccgcgcctgatttgcgatagccgcgtgctggaacgctatctgctggaagcgaaagaagcggaaaacattaccaccggctgcaacgaaacctgcagcctgaacgaaaacattaccgtgccggataccaaagtgaacttttatgcgtggaaacgcatggaagtgggccagcaggcggtggaagtgtggcagggcctggcgctgctgagcgaagcggtgctgcgcggccaggcgctgctggtgaacagcagccaggtgaacgaaaccctgcagctgcatgtggataaagcggtgagcggcctgcgcagcctgaccaccctgctgcgcgcgctgggcgcgcagaaagaagcgattagcccgccggatgcggcgagcgcggcgccgctgcgcaccattaccgcggatacctttcgcaaactgtttcgcgtgtatagcaactttctgcgcggcaaactgaaactgtataccggcgaagcgtgccgcaccggcgatcgc